(19) United States
(12) Patent Application Publication
(10) Pub. No.: US 2024/0293252 A1
(43) Pub. Date: Sep. 5, 2024

(54) Adaptive Garment for Ostomate

(71) Applicant: Mary Jo Ellen Traversone, Lockport, NY (US)

(72) Inventor: Mary Jo Ellen Traversone, Lockport, NY (US)

(21) Appl. No.: 18/442,806

(22) Filed: Feb. 18, 2024

Related U.S. Application Data
(60) Provisional application No. 63/436,496, filed on Mar. 1, 2023.

(51) Int. Cl. A61F 5/449; A61F 5/44

(52) U.S. Cl. CPC A61F 5/449; A61F 5/4408

(57) ABSTRACT

Disclosed herein is “Adaptive Garment for Ostomate” (underwear, shirt, bodysuit, bathing suit) which is a facsimile of normal clothing providing access point(s) for the pass through of an ostomy bag/appliance in which said invention provides a barrier between the ostomy appliance and the skin along with the method of determination of garment access point(s). The embodiment of the invention results in the garment positioned between the body and the ostomy appliance, promoting personal wellbeing, privacy, modesty and euthermia and access to the stoma and the stoma appliance. The garment is worn over the shoulders and under the ostomy appliance to reduce irritation, help maintain body temperature and foster a sense of normalcy. The garment is preferably configured to attain maximum stretching in the vertical direction allowing for greater comfort and range of motion.

### BACKGROUND/SUMMARY

### Field of the Invention

The present invention relates to a garment that is worn by Ostomates when ostomy/colostomy/urostomy bag is in use. The invention relates to an undergarment with an opening allowing access to ostomy appliance through the adaptive garment, passthrough of ostomy appliance outside adaptive garment and removal of ostomy bag while providing additional range of motion and method to determine placement of the access point.

### Background of the Invention

Ostomy surgery (Colostomy, Ileostomy and Urostomy) is a procedure which leaves an opening called the stoma in the abdominal wall of the patient. Patients who undergo such procedure need to use an ostomy bag/appliance to collect bodily waste/discharge. The ostomy bag can be permanent or temporary. The procedure greatly changes the lives of patients who undergo this surgery. There is a need for garments to normalize the lives of the ostomy patient. Garments and appliances currently in production do not facilitate range of motion or promote thermoregulation. This invention will promote the Ostomate's ability to perform activities of daily living.

The present invention provides a one-piece undergarment with an opening provides access for pass through of the ostomy appliance.

### SUMMARY OF THE INVENTION

This Summary is provided to introduce a selection of concepts in a simplified form that is further described below in the Detailed Description Of The Invention. This Summary is not intended to identify key aspects or essential aspects of the claimed subject matter. This Summary is not intended for use as an aid in determining the scope of the claimed subject matter.

The present application discloses an adaptive undergarment that is worn between the ostomy appliance and the epidermis. The ostomy appliance is attached to a flange that is placed over the stoma area. The adaptive garment provides a barrier between the skin, ostomy bag, ostomy belt and other ostomy appliance parts.

The objective of this invention is to provide the ostomate an adaptive garment that provides comfort, reduces dermal breakdown, acts as a barrier when ostomy belt and or ostomy bag is in use, reduces perspiration and helps to maintain body temperature while increasing range of motion.

Another object of this invention is to provide an undergarment constructed from fabric which may comprise one or more layers, that consists of cotton, polyester, spandex, cotton-poly blend, jersey-knit cotton, micro-merino wool, which helps maintain body temperature. However, it will be understood that any type of fabric, whether man-made or natural, coated, raw fiber, disposable or re-usable, may be used without departing from the spirit of the invention.

It is another object of this invention to provide a protective layer between the skin and the ostomy belt, which is used for abdominal wall support to reduce hernias.

The garment consists of an access point sewn into the garment for pass through of the ostomy bag. The opening eliminates the need for a separate urostomy tube hole. The garment is adapted to allow access to the abdominal area of the user.

Another object of this invention is to provide a level of discreetness for those who have undergone an ostomy procedure.

These together with other objects and advantages will become subsequently apparent, reside in the details of the invention as more fully described and claimed, reference being had to the accompanying drawings forming a part thereof, wherein line numerals refer to parts throughout.

### DESCRIPTION

### Brief Description of the Drawings

The novel feature of the invention, as well as the invention itself, both as to its structure and its operation/location will best be understood from the accompanying drawings, taken in conjunction with the accompanying description.

FIG. 1 is a front view of the ostomy garment reflecting the preferred embodiment of the invention as worn by the user of the ostomy appliance.

FIG. 2 is a side view of one type of ostomy appliance suitable for use with the present invention.

FIG. 3 is a front view of a second representation of the invention. To accommodate appliance in various positions relative to the wearer's body, location of access point can be adapted to accommodate both left and right stoma placement.

### DETAILED DESCRIPTION OF THE INVENTION

What follows is a description of various aspect, embodiment and/or examples in which the invention may be practiced. Reference will be made to the attached drawing and the information included in the drawing is a part of this detailed description. The aspects, embodiments and/or examples described herein are presented for exemplification purposes, and not for limitation purposes. Structural and/or logical modifications could be made by someone of ordinary skill in the art without departing from the scope of the invention.

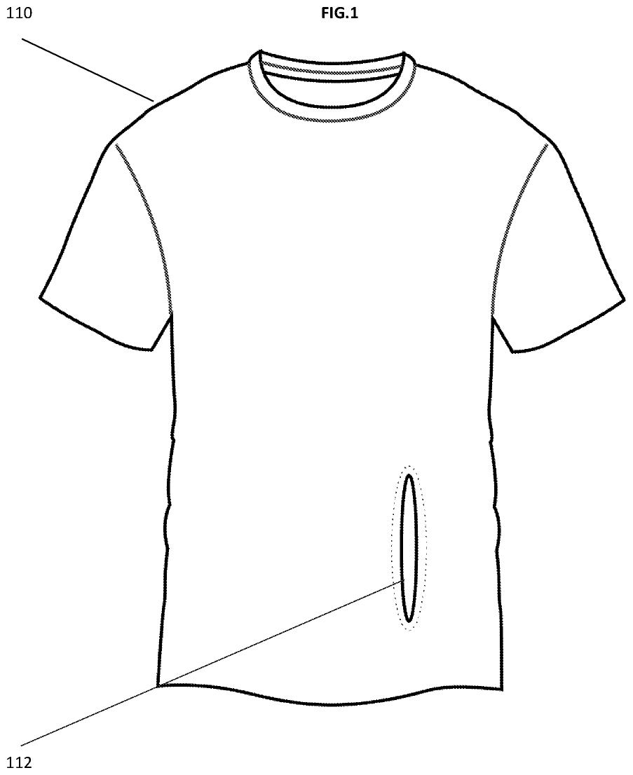

FIG. 1 shows a full-frontal view of the undergarment (110) according to the present invention. As shown in FIG. 1 the present invention is comprised of an opening (112) on a garment (110) that is positioned vertically and for wearer needs could be positioned horizontally or any direction thereof.

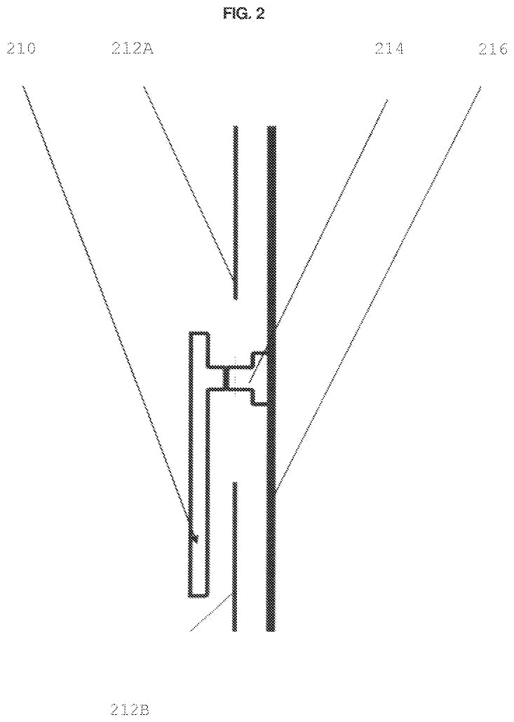

As Shown in FIG. 2 Reflects a side view of the invention providing a layer of fabric between the wearer's skin and ostomy appliance. The body of the wearer (216) is protected by the present invention (212A/212B) with the ostomate's flange (214) and ostomy bag/appliance (210) inserted through the access point.

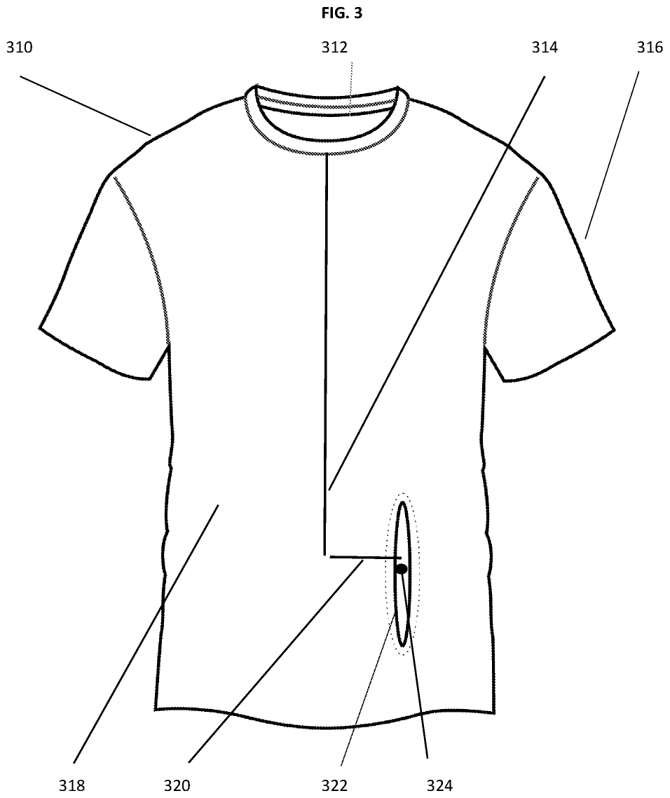

As shown in FIG. 3 For ease of construction, the garment (310) is symmetrical about the longitudinal midlines, although this is not intended to present a constraint on the design. The garment has the appearance of an everyday undergarment, complete with sleeves (316), neck hole (312) and garment body (318). The garment may also be sleeveless without departing from the spirit of the invention, but preference is given to the incorporation of the sleeves (316) for purposes of retaining the patient's body heat. The undergarment can be short or long sleeved or of body suit style. Furthermore, the semi-fitted nature of the garment (310) does not allow for significant volume of air-flow between the patient's body, garment body (318) and the ambient environment. FIG. 3 also reflects method to determine placement of access point (322) which is calculated by measurement of distance from the suprasternal notch and pelvic area (314), in relation to the stoma (324). The distance from midline intersection to the stoma (324) (left or right side) to be identified. (3) This sizing method can be used for all sizes/ages from infant to adult.

The present invention seeks to remedy the problems created by existing garments by amending the garment and creating improved garment functionality through use of materials which have the requisite insulative and heat-retentive characteristics. The invention provides the ostomy patient use of normal clothing for personal comfort, to promote psychological and physiological wellbeing and provide a greater range of motion by use of access point in the undergarment.

Adaptive access point to be surged/sewn to allow for long term use of undergarment. Size of adaptive access point(s) will vary based on age/size of the user. The access point can be circular, oval, oblong, rectangular, triangular, diamond, oval shaped or of variation.

Adaptive access point to allow range of motion/greater flexibility. Adaptive opening will vary based on size of ostomate (generally 3″-12″).

The Adaptive Undergarment for Ostomate is to be worn under ostomy bag to reduce irritation, skin breakdown, reduce perspiration, maintain body temperature, and provide comfort to the wearer.

For those patients that struggle with maintaining body temperature, the Adaptive Undergarment For Ostomate can be tucked into pants, skirts, etc. then layered with an outer shirt for fashion purposes. Undergarment can be of tank top or T-Shirt style (racer back, long sleeve, short sleeve, tank bra, etc).

The undergarment of the present invention is designed for both male and female users and the invention works for multiple types of drainable and closed ostomy bags. Furthermore, the access point can fit bags with variable lengths, shapes, and sizes.

While many of the fundamental characteristics and features of the undergarment have been described herein, with reference to embodiment thereof, a latitude of modification, various changes and substitutions are intended in the foregoing disclosures, and it should be apparent that in some instances some features of the invention will be employed without a corresponding use of other features without departing from the scope of the invention. Consequently, all such modifications and variations are included within the scope of the invention as defined herein.

1. The one-piece ostomy undergarment of claim 1, characterized in that said undergarment comprising of an access point(s) to one or more stoma locations.

2. The one-piece undergarment of claim 1, characterized in that size and positioning of access point to vary based on wearer size/age and location of the stoma (s).

3. The one-piece undergarment of claim 1, characterized in that access point to be sewn or surged to stabilize fabric which allows for long term use.

4. The one-piece undergarment of claim 1, provides additional range of motion for the wearer, helps maintain body temperature, reduces dermal irritation and fosters a sense of normalcy.

5. The one-piece garment of claim 2, characterized in that said placement of access point(s) to be determined by:
1. FIG. 3A line starting from the suprasternal notch to the inferior edge of the shirt (314)
2. FIG. 3A line perpendicular to line 1 (320) that bisects the stoma (324).
